(19) United States
(12) Patent Application Publication
(10) Pub. No.: US 2008/0125631 A1
(43) Pub. Date: May 29, 2008

(54) Method for measuring the rate of transepidermal water loss

(76) Inventor: Robert Erich Imhof, Kent (GB)

Correspondence Address: Bartlett Sherer, 20 Oakmont Circle, New Freedom, PA, 17349, US

(21) Appl. No.: 12/011,087

(22) Filed: Jan. 24, 2008

Related U.S. Application Data
(63) Continuation-in-part of application No. 10/530,780, filed on Apr. 7, 2005, now abandoned.

(30) Foreign Application Priority Data
Oct. 8, 2002 (GB) 02 232 74.2

(51) Int. Cl. A61B 5/00

(52) U.S. Cl. 600/307

(57) ABSTRACT

A method for measuring the rate of transepidermal water loss TEWL using a device for measuring TEWL which comprises:

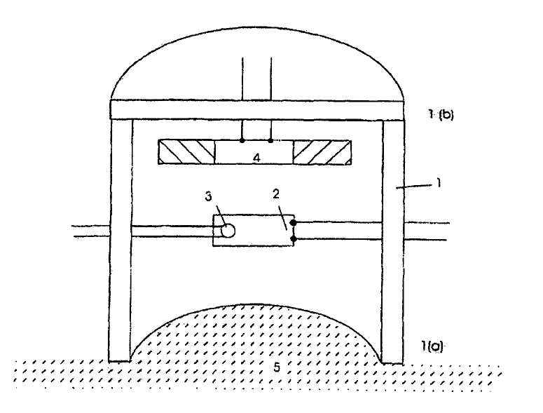

(a) a measurement chamber with a single opening at one end, which opening is adapted to be placed against a test surface of skin;
(b) means to measure the density of water vapour within the measurement chamber; and
(c) an air agitating means positioned within the measurement chamber;
which method comprises:
(i) purging the measurement chamber of the device;
(ii) placing the open end of the measurement chamber against a test surface of skin;
(iii) measuring the rate of change of the density of water vapour within the measurement chamber; and
(iv) operating the agitating means whilst measuring changes of the density of water vapour within the measurement chamber to near uniformly mix water vapour from the test surface and air trapped in the measurement chamber.

### REFERENCE TO PREVIOUS APPLICATIONS

This is a Continuation-in-Part of U.S. patent application Ser. No. 10/530,780 based on PCT Patent Application No. PCT/GB2003/004365 filed 8 Oct. 2003 and claiming priority from UK Patent Application No. 02 232 74.2 filed 8 Oct. 2002.

### FIELD OF THE INVENTION

The present invention relates to a method for measuring the rate of transepidermal water loss TEWL.

### BACKGROUND OF THE INVENTION

TEWL is important in the evaluation of the efficiency of the skin-water barrier. Damage to the skin resulting from various skin diseases, burns and other causes can affect the TEWL and measurement of the TEWL can indicate such damage and possibly its early onset or response to treatment. It therefore has a use in clinical diagnosis.

As the TEWL is a measure of the effectiveness of the skin-water barrier, its measurement is important in assessing skin damage caused by interaction with external substances including soaps, detergents and industrial chemicals. Pre-maturely born infants do not have a fully formed stratum corneum and TEWL measurements can monitor its formation and warn of dehydration due to excessive water loss. TEWL is also used more generally in testing the effect of pharmaceutical and cosmetic products applied to the skin.

TEWL measurement is a special case of the more general problem of measuring the water vapour flux density emanating from a small area of surface—the test surface. Devices and methods for measuring this quantity can conveniently be divided into two categories, namely:—

(Di) Time-series methods that can measure changes in the density of water vapour over prolonged periods of time. Time series methods include the open chamber diffusion gradient method described in GB patent 1 532 419 (Nilsson); flowing gas methods such as used in equipment manufactured by Skinos Co Ltd, Japan; and the closed chamber condenser method described in PCT/GB99/02183 (Imhof). Time-series methods all incorporate a means of preventing the accumulation of water vapour from the test surface within their measurement chambers, this being an essential requirement for continuous measurement over a prolonged period of time.
(Dii) Single-value methods that can only measure changes in the density of the water vapour over a short interval of time, typically of the order of one minute or less depending on the size of the measurement chamber. These methods use closed measurement chambers in which the water vapour emanating from the test surface is trapped without any means of escape or removal. At the end of the measurement, the water vapour that has accumulated in the measurement chamber needs to be removed in some way before the next measurement can be attempted. Apparatus which use single-value methods include the Vapometer manufactured by Delfin Technologies Ltd, Finland and described in WO 01/35816 A1; the instrument described by Tagami et al in Skin Research & Technology, Vol. 8, pp 7-12, 2002; and the dynamic porometer such as the instrument manufactured by Delta-T Ltd, UK.

One of the most firmly held beliefs in measuring TEWL is that the best measurement accuracy is achieved by ensuring that the measurement device does not disturb the boundary layer of still air above the skin. However, this is in conflict with the need to ensure that the sensors used for determining the TEWL, which may be sited some distance away from the skin, give readings that are accurately representative of the TEWL.

In L.-O Lamke, G. E. Nilsson and H. L. Reithner, Acta Chir Scand, 143, 279-84, 1977, a device is disclosed for measuring the evaporative water loss from abdominal cavities during surgery. Because of the relatively large volume of the measurement chamber the device is provided with a low speed fan which is intended to try and make the composition in the measurement chamber substantially uniform whilst, at the same time, trying to minimise disturbance to the boundary layer above the tissue surface, which would materially change the evaporative water loss from it. It should be noted that the aim of this apparatus was to measure evaporative water loss, not TEWL. Of course, it can be used to measure TEWL as the authors mention but it is not efficient for this purpose.

The problem with existing single-value methods of TEWL is that the same instrument can give a range of readings even when used with identical sources. The inventor believes that this is because the composition of the vapour in the measurement chamber is not consistently uniform. The inventor believes that this is due partly to diffusion and partly to air movement which is uncontrolled and caused by natural convection, for example, driven by the temperature difference between the skin and the air trapped in the measurement chamber.

The present invention addresses this problem from an entirely different perspective. In particular, the inventor takes the view that TEWL is determined by the rate at which moisture passes through the skin and consequently a TEWL measurement by evaporimetry requires a sufficiently rapid surface evaporation to ensure that this moisture does not accumulate on the skin surface. It is therefore not necessary or desirable to keep either the boundary layer adjacent the skin or the atmosphere within the measurement chamber still. Indeed the inventor recommends that the air within the measurement chamber is vigorously agitated.

### SUMMARY OF THE INVENTION

In its broadest aspect the present invention provides:

a method for measuring the rate of transepidermal water loss TEWL using a device for measuring TEWL which comprises:
(a) a measurement chamber with a single opening at one end, which opening is adapted to be placed against a test surface of skin;
(b) means to measure the density of water vapour within the measurement chamber; and
(c) an air agitating means positioned within the measurement chamber;
which method comprises:
(i) purging the measurement chamber of the device;
(ii) placing the open end of the measurement chamber against a test surface of skin;
(iii) measuring the rate of change of the density of water vapour within the measurement chamber; and
(iv) operating the agitating means whilst measuring changes of the density of water vapour within the measurement chamber to near uniformly mix water vapour from the test surface and air trapped in the measurement chamber.

Advantageously, said agitating means has a throughput of not less than 0.11/min.

Preferably, said method includes the step of measuring the relative humidity within the measurement chamber.

Advantageously, said method includes the step of measuring the temperature within the measurement chamber.

Preferably, the step of measuring the density of water vapour within the measurement chamber is carried out with at least one sensor positioned within the measurement chamber or outside it, which sensors is/are able to measure quantities from which the density of water vapour within the measurement chamber can be calculated.

Advantageously, said method includes the step of measuring the density of water vapour within the measurement chamber by measuring the absorption of infrared radiation of suitable wavelength by the water vapour.

Preferably, said agitating means comprises a fan.

Advantageously, said fan has a diameter of about 4 mm.

Preferably, said fan is rotated at about 15,000 revolutions per minute.

Advantageously, said fan has a power consumption of about 0.2 w.

The measurement chambers of the single-value methods cited in (Dii) above need to be purged to remove any water vapour accumulated during a previous measurement. This can be done by injecting a small quantity of dry gas prior to a measurement, as in the dynamic porometer of Delta-T Ltd, UK, for example. This method of purging has the disadvantages of size, weight and complexity associated with the gas purging system. Another method, used with the Vapometer manufactured by Delfin Technologies Ltd for example, is to move the measurement wand incorporating the measurement chamber rapidly through ambient air, such movement causing the measurement chamber to be purged through turbulent mixing with ambient air. This has the disadvantage of lack of control and reproducibility.

By using the agitating means in an appropriate manner purging can be achieved reliably, consistently and quickly.

Accordingly, the present invention also provides a method in which said purging is provided by said agitating means.

The agitating means is preferably operated to purge the measurement chamber, to make it ready for the next measurement in as short a time as possible. Purging with ambient air can occur before, after or both before and after each measurement, to provide reproducible conditions for each measurement. The agitation needs to be vigorous enough to cause rapid turbulent mixing to occur between the trapped humid air in the measurement chamber and ambient air in the vicinity of the chamber opening. Rapid in this context means faster than by the conventional means of moving the wand through the air (20-30 seconds in the case of the Delfin instrument), and preferably faster than the time during which the measurement face is in contact with the test surface. Thus a purging time of 10 seconds or less is desirable.

Preferably, the agitating means has a throughput at least 6 times the volume of said measurement chamber per second to enable said measurement chamber to be purged rapidly.

The means to measure the water vapour density within the chamber can be sensors positioned within the chamber which are able to measure quantities from which the density of water vapour within the chamber can be calculated. The quantities from which the density of water vapour can be determined include relative humidity and temperature etc. The sensors need not be deployed wholly inside the measurement chamber. Deployment on the outside of the measurement chamber, as described in Patent Application PCT/GB 2003/000265, may be more convenient.

Alternative means of measuring water vapour density in the measurement chamber can be used such as a sensor based on measuring the absorption of infrared radiation of suitable wavelength by the water vapour. If the temperature of the air within the measurement chamber remains nearly constant throughout a measurement sequence, then the temperature sensor within the measurement chamber may be dispensed with.

Preferably the air agitation means is a mechanical device such as a fan; however alternative means of agitating the air in the measurement chamber can be deployed, with the motive power supplied by electrical, pneumatic or other means, providing rotary, reciprocating or other motion to an agitator propeller or paddle. The source of motive power can be situated either inside or outside the measurement chamber. If the source of motive power is situated on the outside of the measurement chamber, then it can conveniently be coupled to the agitator inside the measurement chamber by means of a shaft, electromagnetic or other form of coupling.

Preferably, the measurement chamber will be of circular cross-section with an internal diameter of from 5 to 15 mm with 6 mm being preferred.

Advantageously the internal length of the measurement chamber will be from 10 to 30 mm with 10 mm being preferred.

In use, the open end of the equipment is placed against the test surface, e.g. skin. The agitation of the air may be active before contact is made with the test surface, so that the chamber is purged with ambient air immediately before the measurement. The sensor readings from which the density of the water vapour and hence the flux density can be determined are then made. During these measurements, the agitation of the air within the chamber is active, to promote efficient evaporation from the test surface and to mix the water vapour emanating from the test surface with the trapped air to near-uniform properties of humidity and temperature. When the measurement is finished, the agitation of the air in the measurement chamber needs preferably to be active, so that the chamber is purged rapidly of the water vapour accumulated during the measurement.

The readings from the sensors of typically relative humidity and temperature can be used to calculate the density of water vapour within the measurement chamber. The agitation ensures that the water vapour from the test surface is actively and rapidly mixed with the air enclosed in the measurement chamber and that the vapour density is therefore uniform throughout. The positioning of the sensors within the measurement chamber is therefore not critical.

If uniform mixing of the water vapour entering the measurement chamber from the test surface and the air trapped within it is assumed, the water vapour flux density emanating from the test surface can be calculated from the rate of increase of water vapour density in the measurement chamber using Eq. 1

$$J = \frac{V}{A} \cdot \frac{\partial ρ}{\partial t} \qquad Eq . 1$$

where
J is the water vapour flux density
V is the volume of the measurement chamber
A is the open area of the measurement chamber in contact with the test surface
ρ is the water vapour density within the measurement chamber

The assumption made in the derivation of Eq. 1 is that the water vapour emanating from the test surface would remain as water vapour within the measurement chamber. This condition is satisfied as long as (a) the relative humidity everywhere within the measurement chamber remains below 100%, and (b) the materials within the measurement chamber which come into contact with water vapour are not hygroscopic. If condition (a) is not satisfied, then condensation of water vapour to liquid water may occur. It is therefore important to ensure that the measurement is terminated and the measurement chamber is removed from the test surface well before such saturation conditions are reached. If condition (b) is not satisfied, then a quantity of water vapour may be lost temporarily through surface adsorption. Conversely, previously adsorbed water may be desorbed when the humidity in the measurement chamber is low. These processes may lead to measurement errors such as “memory effect” or hysteresis.

According to Eq. 1, the water vapour flux density can be calculated from the rate of increase of water vapour density in the measurement chamber. If the flux density is constant, then this rate of increase is constant. It can then be calculated, for example, from the difference between two vapour density values calculated from readings taken at two separate times, or from a least-squares calculation to a series of vapour density values calculated from readings taken over an appropriate time interval. Changes of water vapour flux density during a measurement manifest themselves as changes of the rate of increase of water vapour density in the measurement chamber.

Eq. 1 is not specific to any particular geometry of measurement chamber or deployment of sensors within it. Therefore any convenient shape can be used e.g. cylindrical, rectangular parallelepiped, prism, etc. However, its main dimensions of volume V, and open area A in contact with the test surface are important parameters that can be adjusted to a particular measurement application. The parameter A is the area of test surface over which the mean flux density is calculated. The ratio A/V determines the sensitivity of measurement. In addition, A/V is inversely proportional to the length of time taken before saturation conditions are approached and therefore the maximum duration of the measurement for a given value of flux density.

A suitable and convenient method of measuring the density of water vapour within the measurement chamber is by using common sensors of relative humidity and temperature, the two sensors acting together to measure these two properties at essentially the same location. A suitable and convenient choice of relative humidity sensor includes those based on a change of capacitance or a change of electrical conductivity etc, which are widely commercially available. A suitable and convenient choice of temperature sensor includes the conventional thermocouple and thermistor, which are widely commercially available. Alternatively a composite sensor can be used which simultaneously measures relative humidity and temperature so that one such composite sensor can produce the required signals.

The water vapour density can be calculated from measured values of relative humidity and temperature using the well known relationship

$$ρ = \frac{RH \%}{100} \cdot {ρ}_{S} ( θ ) \qquad Eq . 2$$

where
RH % is the percentage relative humidity
θ is temperature
ρₛ is the saturation vapour density

The saturation vapour density can conveniently be computed from an empirical parameterisation such as that of P. R. Lowe, J. Appl. Meteorol., Vol. 16, pp 100-3, 1977.

In use, the open end of the measurement chamber is placed against the test surface and a start-signal is sent to the processor to initiate a measurement sequence. This start-signal is conventionally and conveniently generated manually by the user actuating a switch such as a push-button on the handle of the measurement wand or a foot switch. Alternatively, an automatic means of generating a start-signal can be deployed. One example is to sense the increase of relative humidity or vapour density in the measurement chamber against a reference value provided by similar sensors used for measuring ambient conditions. Another example is to deploy a light sensor such as a photodiode in the measurement chamber to generate a start-signal when the light level decreases below a pre-set value as the measurement chamber makes contact with the test surface.

Once the start-signal has been received, readings from the sensors are taken periodically by a processor in order to record the time change of the signals. The measurement sequence is terminated and the contact between the measurement chamber and the test surface is broken after a predetermined criterion or set of criteria are satisfied. Most importantly, the measurement must be terminated when the relative humidity within the measurement chamber reaches a pre-determined level. This level is chosen to be high enough to allow the measurement to be taken but low enough to prevent condensation from occurring. Other criteria that can be used to terminate a measurement in advance of this include a pre-set measurement time or a pre-set measurement precision.

In a typical pharmaceutical or cosmetic procedure test patches of substances being tested will be applied to the arms of a patient. Six to eight patches would be typical. The TEWL through each patch-is measured sequentially and the results recorded. It is in this environment that the major advantages of the preferred methods become apparent. In particular, the measurement chamber can be purged quickly and efficient before use. The TEWL reading obtained will be repeatable. When the reading has been obtained the measurement chamber can be purged in a known and repeatable manner which can also be very fast. The whole testing process can be carried out in a significantly shorter time than was previously possible thereby not only saving the time of the volunteer and the person carrying out the test but also giving a better comparative assessment of the TEWL of the substances which can change over time.

### BRIEF DESCRIPTION OF THE DRAWING

The invention is described with reference to the accompanying drawing which is a side view of an embodiment of a device for carrying out the preferred method of the invention.

### DETAILED DESCRIPTION OF THE PREFERRED EMBODIMENT

Referring to the drawing there is shown a measurement chamber 1 in the form of a hollow cylinder which has an open end 1a and a closed end 1b. The prototype measurement chamber 1 was made from PTFE and had an internal diameter of approximately 6 mm and an overall internal height of approximately 10 mm. However, it is anticipated that measurement chambers will typically have an internal diameter from 5 to 15 mm and an overall internal height of from 10 to 30 mm. It is also anticipated that the measurement chamber 1 could be made from any dense plastics or other material that does not absorb or adsorb significant quantities of water and which does not irritate the skin on contact therewith.

Inside the measurement chamber 1 are a capacitative relative humidity sensor 2 and a thermistor 3 that measure the relative humidity and temperature respectively at substantially the same location. The sensors could also have been located in the curved wall of the cylindrical measurement chamber. The outputs of the capacitative relative humidity sensor 2 and the thermistor 3 are fed to a computer not shown. Also inside the cylinder is a fan 4 to agitate the air and cause uniform mixing of the enclosed water vapour and air. In the prototype device, the fan 4 had a diameter of 4 mm and was powered by a DC electric motor to rotate at speeds up to 15000 RPM at a power consumption of up to 0.2 Watt. The air flow in this implementation was substantially impeded by the proximity of the wall of the measurement chamber producing mainly turbulence within the measurement chamber 1 rather than directed air flow. This turbulence causes effective mixing of TEWL water vapour with enclosed air during measurements. After measurements, the rate of exchange of air with the outside air (ie the purging rate) of the prototype device was estimated to be of the order of 0.1 litres/min or of the order of 6 changes of air every second.

To measure the water vapour flux density from the test surface 5 such as the skin of a person, the open end 1a is placed against the skin, so that the measurement chamber becomes closed trapping air which mixes with vapour from the skin. At the same time as the measurement chamber makes contact with the test surface or immediately afterwards, a start-signal is sent to the computer to initiate a measurement sequence. The means by which this start-signal is generated is not shown. The computer is programmed with a program so that the output from the sensors 2 and 3 are converted to a reading in the desired form, e.g. water vapour flux density from the surface. A graphical representation of the readings or quantities derived from the readings may also be used to verify that the underlying assumptions hold true and that the measurement is valid. The fan 4 is operated during the measurements by the capacitative relative humidity sensor 2 and the thermistor 3 are taken to ensure that the vapour is mixed rapidly with the trapped air to produce a vapour-air mixture of near-uniform humidity and temperature.

After a measurement and before the humidity within the measurement chamber 1 has increased to a value where condensation might occur, the contact between the measurement chamber 1 and the test surface is broken and ambient air is mixed with the previously trapped air with the help of the fan 4, in order to restore the humidity and temperature conditions within the measurement chamber 1 to those of ambient air.

In the implementation described, only one relative humidity sensor and one temperature sensor is required, thus simplifying the construction. This does not preclude the use of more sensors, however. The use of additional sensors would allow more precise calculations of water vapour flux density to be performed, if the distribution of water vapour within the measurement chamber 1 were not perfectly uniform. It may also be convenient to incorporate additional sensors in the equipment outside the measurement chamber, to measure ambient temperature, ambient humidity, skin temperature, etc.

The measurement chamber 1 can conveniently be incorporated in a hand-held wand or with a convenient handle etc.

The equipment and method can be used to measure any vapour flux density from a test surface although, when the vapour is not water vapour, the sensors are chosen accordingly.

The device and method can be used with any test surface. Apart from skin, the equipment can be used to measure water vapour flux from plant leaves, etc. The cylinder is the common geometry of measurement chamber for such instruments, but any convenient shape can be used, e.g. rectangular parallelepiped, prism, etc.

In a typical commercial environment, 8 patches of various cosmetic substances are applied to the forearm of a volunteer. Prior to the first measurement the measurement chamber 1 is purged by running the fan 4 for 10 seconds. The open end of the measurement chamber 1 is then placed on the patch of skin under test and the TEWL measured with the fan 4 continuing to run. The measurement typically takes 10 seconds. At the end of the measurement, the measurement chamber 1 is taken off the patch of skin and held for just 10 seconds in the surrounding air with the fan 4 rotating. This is sufficient to purge the measurement chamber 1 which is then immediately applied to the second patch of skin to be tested and the process repeated. It will be appreciated that the entire process takes significantly less time than was previously necessary. Furthermore, if a measurement is repeated on a given patch of skin, the same or a very close reading can be obtained.

Although the internal diameter of the measurement chamber 1 in the preferred embodiment was 6 mm and the internal length 10 mm the measuring chamber could typically be from 5 to 15 mm in internal diameter and from 10-30 mm in internal length.

The preferred fan 4 had a throughput of 0.1 litre/min. In experiments we found that in the context of the measuring chamber 1 the throughput should preferably not be allowed to drop below 0.1 litres/min because this would slow the purging and undesirably delay the next measurement. Increasing the throughput of the fan 4 seemed to have no noticeable detrimental effect on the measured TEWL.

In experiments with the prototype device we found that with the fan 4 switched off the coefficient of variation (ie: 100*standard deviation/mean) between successive measurements was about 10%. With the fan 4 switched on the coefficient of variation reduced to about 6%. This demonstrated the higher repeatability of the measurements.

1. A method for measuring the rate of transepidermal water loss TEWL using a device for measuring TEWL which comprises:
(a) a measurement chamber with a single opening at one end, which opening is adapted to be placed against a test surface of skin;
(b) means to measure the density of water vapour within the measurement chamber; and
(c) an air agitating means positioned within the measurement chamber;
which method comprises:
(i) purging the measurement chamber of the device;
(ii) placing the open end of the measurement chamber against a test surface of skin;
(iii) measuring the rate of change of the density of water vapour within the measurement chamber; and
(iv) operating the agitating means whilst measuring changes of the density of water vapour within the measurement chamber to near uniformly mix water vapour from the test surface and air trapped in the measurement chamber.

2. A method according to claim 1, wherein said agitating means has a throughput of not less than 0.1 l/min.

3. A method according to claim 1, including the step of measuring the relative humidity within the measurement chamber.

4. A method according to claim 1, including the step of measuring the temperature within the measurement chamber.

5. A method according to claim 1, including the step of measuring the density of water vapour within the measurement chamber with at least one sensor positioned within the measurement chamber or outside it, which sensors is/are able to measure quantities from which the density of water vapour within the measurement chamber can be calculated.

6. A method according to claim 5, including the step of measuring the density of water vapour within the measurement chamber by measuring the absorption of infrared radiation of suitable wavelength by the water vapour.

7. A method according to claim 1, wherein said agitating means comprises a fan.

8. A method according to claim 6, wherein said fan has a diameter of about 4 mm.

9. A method according to claim 6, wherein said fan is rotated at about 15,000 revolutions per minute.

10. A method according to claim 6, wherein said fan has a power consumption of about 0.2 w.

11. A method according to claim 1, wherein said purging is effected by said agitating means.

12. A method according to claim 7, wherein said agitating means has a throughput at least 6 times the volume of said measurement chamber per second to enable said measurement chamber to be purged rapidly.

13. A method according to claim 1, wherein said measurement chamber is of circular cross-section and has an internal diameter between 5 and 15 mm.

14. A method according to claim 13, wherein said measurement chamber has an internal diameter of 6 mm.

15. A method according to claim 1, wherein said measurement chamber has an overall length of from 10 to 30 mm.

16. A method according to claim 15, wherein said measurement chamber has an overall length of 10 mm.
